(19) United States
(12) Patent Application Publication
(10) Pub. No.: US 2020/0347906 A1
(43) Pub. Date: Nov. 5, 2020

(54) AUTOMATIC PRESSURE REGULATING PNEUMATIC CYLINDER

(71) Applicant: KEN DALL ENTERPRISE CO., LTD., New Taipei City (TW)

(72) Inventors: Chia-Pao Cheng, New Taipei City (TW); Chih-Hsuan Liang, New Taipei City (TW)

(21) Appl. No.: 16/402,250

(22) Filed: May 3, 2019

(51) Int. Cl. F16F 9/516; F16F 9/02; A61F 2/50

(52) U.S. Cl. CPC F16F 9/5165; F16F 9/0218; A61F 2/50; F16F 2222/126; A61F 2002/5038; F16F 2228/066; F16F 2230/183; A61F 2002/5006; F16F 2232/06

(57) ABSTRACT

An automatic pressure regulating pneumatic cylinder includes: a pneumatic cylinder, arranged with a connecting element for sealing the pneumatic cylinder, a piston slidably configured above the connecting element, a upper air chamber formed above the piston, and a lower air chamber formed between the piston and connecting element, the pneumatic cylinder further configured with first and second air entering passages allowing external air to one-way flow into the upper air chamber and a first guide passage allowing the external air to one-way flow into the lower air chamber; an air flow control unit, embedded inside the connecting element, an exhaust floating piston of the air flow control unit allowed to correspondingly form an air passage for opening or closing through normal speed and rapid displacement of the piston and further cause the internal air of the lower air chamber to generate different air pressure resistance for automatic internal pressure regulation.

### (a) TECHNICAL FIELD OF THE INVENTION

The present invention relates to an automatic pressure regulating pneumatic cylinder, utilizing a plurality of passages for unidirectional suction of external air and one-way exhaust to allow the piston configured inside to automatically adjust the volumes of the upper, lower air chambers and the exhaust speed to a state most suitable for users through the speed of travel of the piston.

### (b) DESCRIPTION OF THE PRIOR ART

The use of pneumatic cylinders on prosthetic joints has become a quite often and common design with the development of medical practice and technology. However, it is necessary to make quite cumbersome and time-consuming adjustments for items such as body type, habits, comfort when they are installed on users, and in the end, the user can have a more comfortable wearing experience.

U.S. Pat. No. 9,180,026 discloses an adjustment-free cushioning air cylinder, using an air pressure chamber, inside which a piston is configured so as to allow the air pressure chamber to be divided into a upper air chamber and lower air chamber, where one end of the piston is configured inside the air pressure chamber, and another end thereof is extended to the outside of the air cylinder. Furthermore a first check valve is configured inside the piston and in connection with the upper air chamber and lower air chamber, allowing air flow to unidirectionally enter the lower air chamber from the upper air chamber, and a upper air way is formed on the air cylinder and in communication with the upper air chamber and lower air chamber; the second check valve is configured inside the upper air way and one end of the second check valve is in communication with the outside and another thereof the upper air chamber, allowing air flow to be unidirectionally flow into the upper air chamber from the outside. In addition, the lower air way is formed on the air cylinder and in communication with the lower air chamber and the outside. In addition, the diameter of the upper air way is larger than that of the lower air way, allowing the air entering rate of the air chamber to be greater than the air discharge rate thereof. Whereby, the complicated internal structures of the conventional air cylinders can be overcome, and the air flowing unidirectionality and the advantage of the air entering amount being greater than the air discharge amount allow the regular air filling to be omitted. With the user's walking habits, the air intake can be automatically adjusted, thereby having a high cushioning performance.

However, the exhaust and intake volume of the check valve of the above adjustment-free cushioning air cylinder must be adjusted manually if only depending the single structure of air entering amount being greater than air discharge amount, but relative to different users, the center of gravity of the walking force, the speed and the applicable regionality will take a longer time to adapt.

### SUMMARY OF THE INVENTION

The present invention proposes an automatic pressure regulating pneumatic cylinder, including: a pneumatic cylinder, arranged with a connecting element for sealing the pneumatic cylinder on lower inside thereof, a piston slidably configured above the connecting element, a upper air chamber formed inside the pneumatic cylinder above the piston, and a lower air chamber formed inside the pneumatic cylinder between the piston and connecting element, an outside of the pneumatic cylinder further configured with a first air entering passage and second air entering passage allowing external air to one-way flow into the upper air chamber and a first guide passage allowing the external air to one-way flow into the lower air chamber and be converted into internal air; an air flow control unit, embedded inside the connecting element and composed of an exhaust floating piston, sealing element, elastic element and exhaust floating piston positioner from top to bottom, the exhaust floating piston allowed to correspondingly form an air passage for opening or closing through normal speed and rapid displacement of the piston and further cause the internal air of the lower air chamber to generate different air pressure resistance, allowing the pneumatic cylinder to achieve automatic internal pressure regulation.

The automatic pressure regulating pneumatic cylinder of the present invention mainly can conform to user's activity states, utilizing the first and second air entering passages one-way inhaling external air to uniformly push the piston downward, and utilizing the first guide passage to allow the external air inside the upper air chamber to flow into the lower air chamber and be converted to internal air. In addition, the air flow control unit is supplemented to automatically adjust the internal air pressure resistance of the pneumatic cylinder, thereby controlling the exhausting action of the lower air chamber. The present invention is easier to be applied than conventional manual precision adjustment.

### BRIEF DESCRIPTION OF THE DRAWINGS

FIG. 1 is a perspective view of the present invention;

FIG. 2 is an exploded view of the present invention;

FIG. 3 is a cross-sectional view of a pneumatic cylinder internal structure of the present invention;

FIG. 4 is a front view of the present invention;

FIG. 5 is a right-side view of the present invention;

FIG. 6 is a schematic view of the present invention, where an exhaust floating piston is moved downward to form an air passage upon generally walking;

FIG. 7 is a schematic view of the present invention, where external air inhaled into a upper air chamber enters a lower air chamber through a first guide passage when a piston is moved;

FIG. 8 is a schematic view of the present invention, where the piston is displaced downward to close the external deflation air passage upon a fast movement; and

FIG. 9 is a schematic view of the present invention, where the automatic pressure relief of the lower air chamber is being performed when the present invention is stationary.

### DETAILED DESCRIPTION OF THE PREFERRED EMBODIMENTS

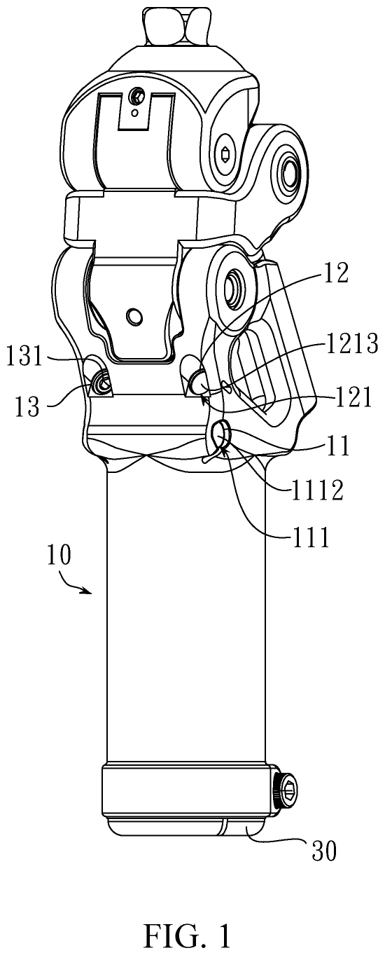

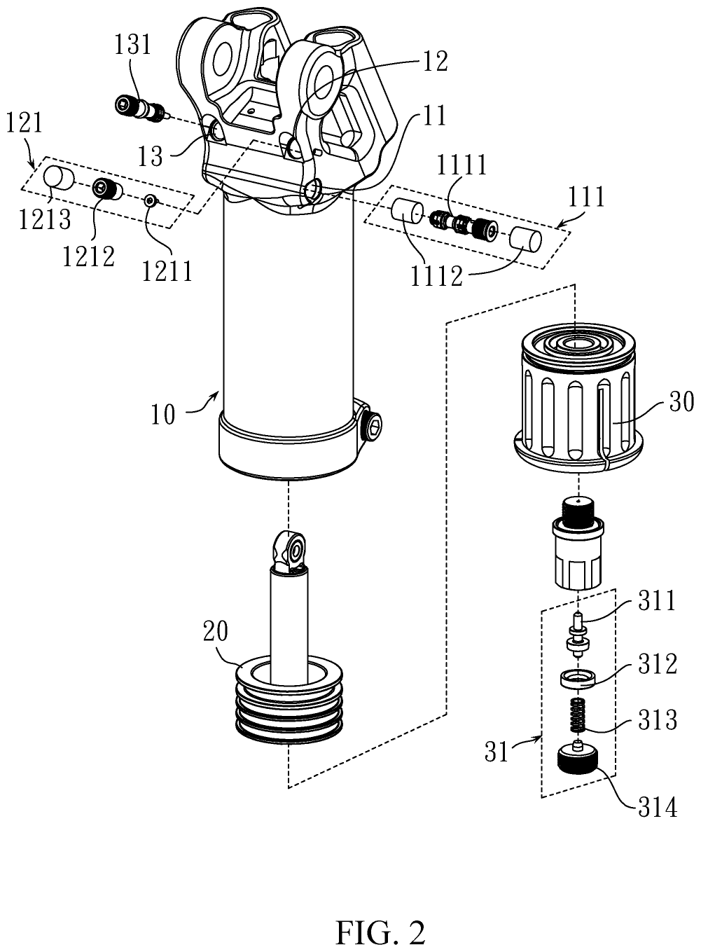

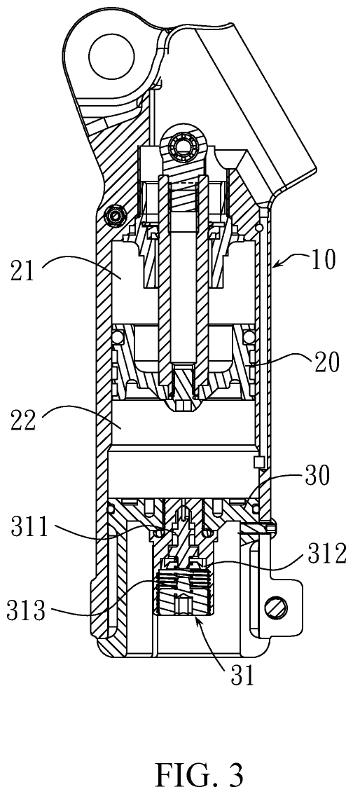

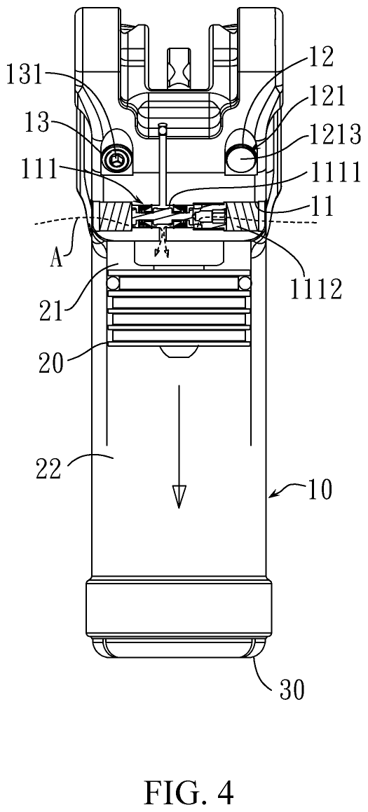

Referring to FIGS. 1 to 9, an automatic pressure regulating pneumatic cylinder includes a pneumatic cylinder 10, inside which a connecting element 30 is configured on the lower side thereof, allowing the pneumatic cylinder 10 to be sealed, and a piston 20 is slidably configured above the connecting element 30, where the pneumatic cylinder 10 is formed with a upper air chamber 21 above the piston 20, and a lower air chamber 22 between the piston 20 and connecting element 30.

A first air entering passage 11 and second air entering passage 12 allowing external air A to unidirectionally flow into the upper air chamber 21 and a first guide passage 13 allowing the external air A to unidirectionally flow into the lower air chamber 22 and be converted into internal air B are further respectively configured on the outside of the pneumatic cylinder 10.

An air flow control unit 31 is embedded in the interior of the connecting element 30 and composed of an exhaust floating piston 311, sealing element 312, elastic member 313, and an exhaust floating piston stopper 314 from top to bottom, where the exhaust floating piston is allowed to correspondingly form an air passage 32 for opening or closing through the piston 20 at normal speed and rapid displacement to further cause the internal air B of the lower air chamber 22 to generate different air pressure resistance, allowing the pneumatic cylinder 10 to achieve the object of having automatic internal pressure adjustment.

Referring to FIGS. 1 to 3 again, the inside of each of the first air entering passage 11, second air enter passage 12 and first guide passage 13 configured on the upper outside of the pneumatic cylinder 10 is configured with objects; the first air entering passage 11 is transversely arranged on the pneumatic cylinder 10 and configured with a first check valve assembly 111 composed of a check valve 111 on two sides of which a filter 1112 is respectively combined; the upper outside of the pneumatic cylinder 10 is longitudinally arranged with the second air entering passage 12, inside which a second check valve assembly 121 is configured, and the second check valve assembly 121 is mainly composed of a check valve 1211, check valve positioner 1212 and filter 1213 in sequence, where the check valve positioner 1212 further can regulate the air flow of the external air A entering the upper air chamber 21; the first guide passage 13 is then symmetrically configured opposite the second air entering passage 12 and configured with a third check valve assembly 131 inside it.

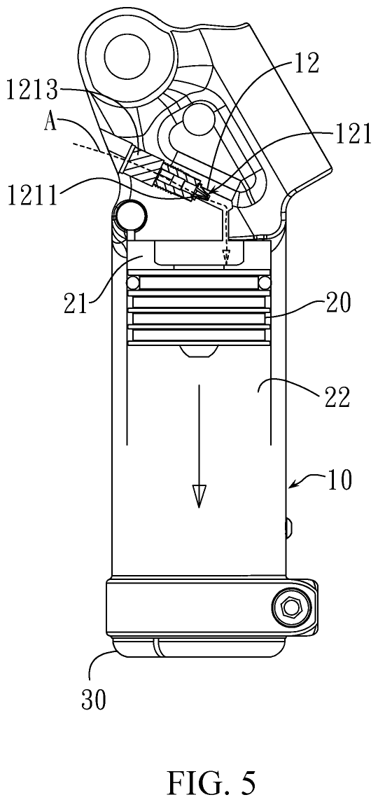

Referring to FIG. 5, the above normal speed state is defined as a user's normal walking speed state; when a user's joint is bent, the piston 20 of the pneumatic cylinder 10 is displaced downward, and the external air (A) will be one-way inhaled into the upper air chamber 21 through the first air entering passage 11 and second air entering passage 12, and at this time, the external air A is converted into the internal air B.

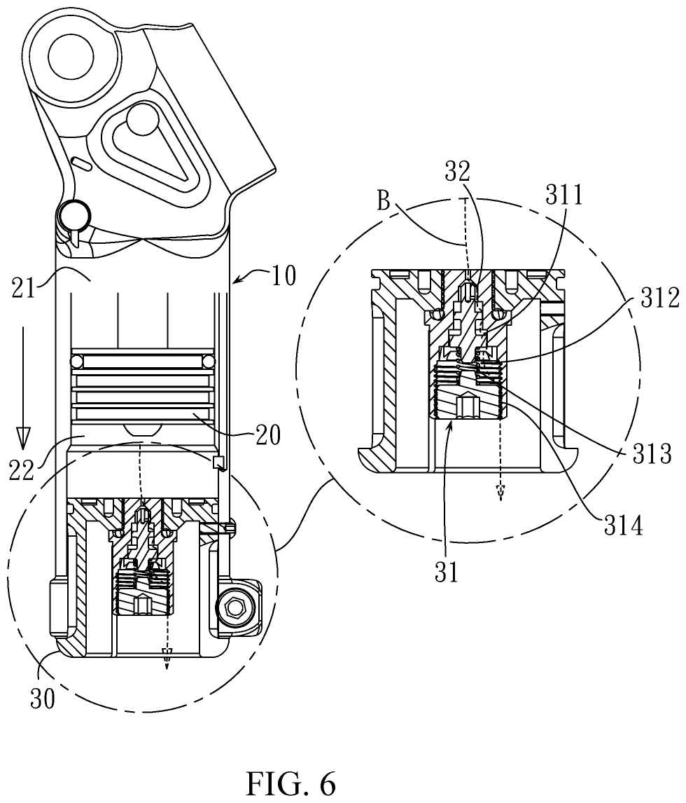

Referring to FIG. 6, the piston will thereafter compress the internal air B inside the lower air chamber 22, allowing the internal air B inside the lower air chamber 22 to slightly push the exhaust floating piston 311 away, thereby forming an air passage 32. But, the exhaust floating piston 311 is not pressed by the sealing element 312 to seal the air passage 32, allowing the internal air B to be discharged to outside the pneumatic cylinder 10 through the gap of the air passage 32, and at this time, the exhaust speed is smaller than the compressed speed of the internal air B inside the lower air chamber 22 such that when the piston 20 is displaced downward, the user will have the feeling of resistance becoming larger because the internal air B is compressed.

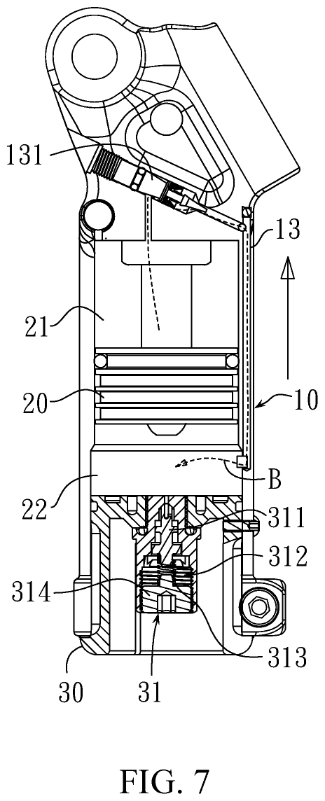

Furthermore, referring to FIG. 7, when the piston 20 is displaced upward, the elastic element 313 will push the exhaust floating piston 311 to return upward to the original position because the elastic element 313 is in connection with the exhaust floating piston 311, thereby sealing the air passage 32, and one-way pushing the external air A of the upper air chamber 21 into the lower air chamber 22 through the third check valve 131 inside the first guide passage 13 and converting it into the internal air B, which completes a motion cycle.

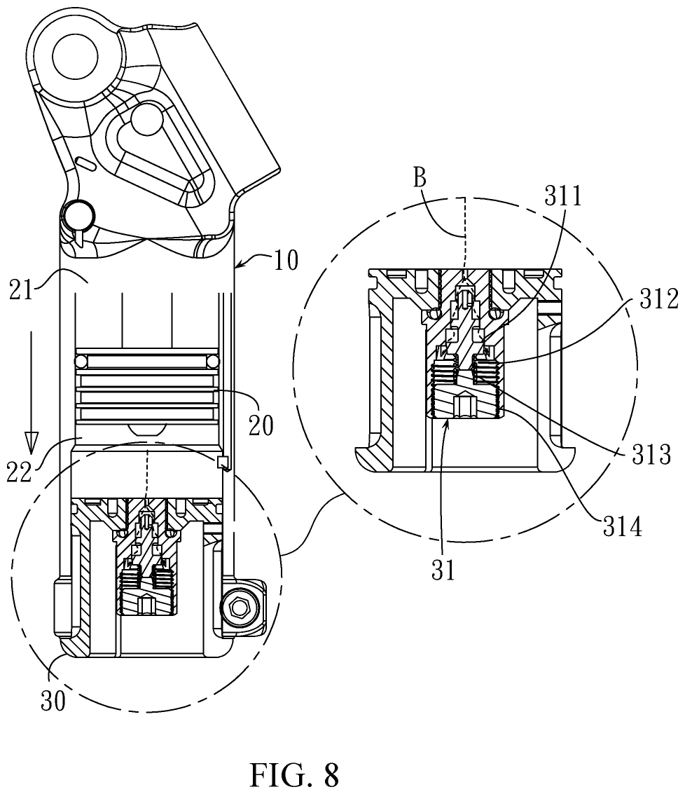

Referring to FIG. 8, the above rapid displacement state is defined as that a user walk or run with a generally fast speed. Namely, the piston 20 inside the pneumatic cylinder 10 will vary with the displacement speed generated from the bending of a user's joint; when the user walk or run fast, the piston 20 will quickly compress the internal air B inside the lower air chamber, allowing the internal air B to completely push the exhaust floating piston to move downward to cause the exhaust floating piston 311 to be in engagement with the sealing element 312 and further cause the lower side of the air passage 32 to be sealed so that the internal air B cannot be discharged. At this time, the internal air B inside the lower air chamber 22 is compressed continuously, thereby providing the piston 20 with sufficient resistance to move fast. In addition, this motion cycle can provide a corresponding air pressure damping adjustment according to the change of the moving speed of the piston 20, so that the pneumatic cylinder 10 can achieve the purpose of automatically adjusting the pressure.

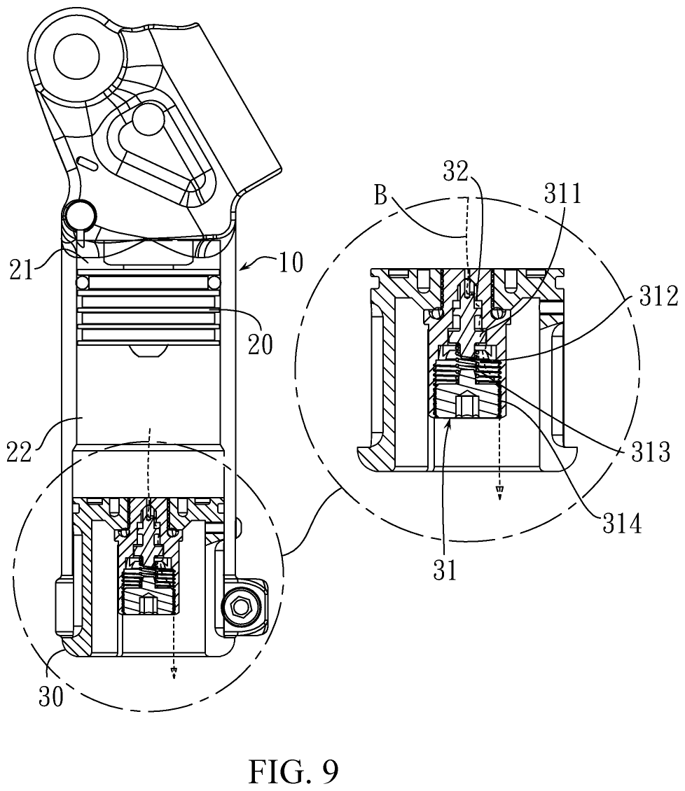

Furthermore, referring to FIG. 9, following the above rapid displacement state shown in FIG. 8, the piston 20 will also stop displacement when the activity is stopped, and the exhaust floating piston 311 will be displaced upward a small amplitude, thereby releasing the engagement thereof with the sealing element 312, allowing a portion of the excess internal air B to exit the pneumatic cylinder 10 through the air passage 32 and the gap to cause the pressure of the lower air chamber 22 to be equal to that outside the pneumatic cylinder 10, thereby returning the pressure inside the pneumatic cylinder 10 to the uncompressed original pressure.

To sum up, the automatic pressure regulating pneumatic cylinder of the present invention one-way inhales the external air A into the upper air chamber 21 inside the pneumatic cylinder 10 through the first air entering passage 11 operated in coordination with the second air entering passage 12; the internal air B inside the lower air chamber 22 is discharged and maintained by completely sealing and opening the exhaust floating piston 311 and sealing element 312 inside the air flow control unit 31 after the piston 20 is stably pushed downward, and the external air A inside the upper air chamber 21 can be guided into the lower air chamber 22 for application through the first guide passage 13 operated in coordination with the moved-upward piston, allowing the present invention to achieve the automatic adjustment of pressure and strengthening use comfort and convenience in normal speed and rapid displacement states.

I claim:

1. An automatic pressure regulating pneumatic cylinder, comprising: a pneumatic cylinder, arranged with a connecting element for sealing said pneumatic cylinder on lower inside thereof, a piston slidably configured above said connecting element, a upper air chamber formed inside said pneumatic cylinder above said piston, and a lower air chamber formed inside said pneumatic cylinder between said piston and connecting element, an outside of said pneumatic cylinder further configured with a first air entering passage and second air entering passage allowing external air to one-way flow into said upper air chamber and a first guide passage allowing said external air to one-way flow into said lower air chamber and be converted into internal air; an air flow control unit, embedded inside said connecting element and composed of an exhaust floating piston, sealing element, elastic element and exhaust floating piston positioner from top to bottom, said exhaust floating piston allowed to correspondingly form an air passage for opening or closing through normal speed and rapid displacement of said piston and further cause said internal air of said lower air chamber to generate different air pressure resistance, allowing said pneumatic cylinder to achieve automatic internal pressure regulation.

2. The pneumatic cylinder according to claim 1, wherein said first air entering passage comprises a first check valve assembly composed of a check valve sandwiched by a filter on each side thereof, and said first check valve assembly is positioned inside said first air entering passage and in combination with said upper air chamber through said first air entering passage with said second air entering passage, allowing said external air to enter said upper air chamber through a movement of said piston.

3. The pneumatic cylinder according to claim 1, wherein said second air entering passage comprises a second check valve assembly composed of a check valve, check valve positioner and filter in sequence, and said second check valve assembly is positioned inside said second air enter passage, allowing said external air to enter said upper air chamber through a movement of said piston.

4. The pneumatic cylinder according to claim 1, wherein said first guide passage comprises a third check valve assembly configured inside said first guide passage, and said first guide passage is symmetrically configured opposite said second air entering passage.
